(19) United States
(12) Patent Application Publication
(10) Pub. No.: US 2024/0341692 A1
(43) Pub. Date: Oct. 17, 2024

(54) PHYSIOLOGICAL INFORMATION ANALYSIS DEVICE AND PHYSIOLOGICAL INFORMATION ANALYSIS METHOD

(71) Applicant: NIHON KOHDEN CORPORATION, Tokyo (JP)

(72) Inventor: Kei KIMURA, Tokorozawa-shi (JP)

(73) Assignee: NIHON KOHDEN CORPORATION, Tokyo (JP)

(21) Appl. No.: 18/624,815

(22) Filed: Apr. 2, 2024

(30) Foreign Application Priority Data
Apr. 11, 2023 (JP) 2023-064123

(51) Int. Cl. A61B 5/00; A61B 5/0205; A61B 5/021; A61B 5/024; A61B 5/145; A61B 5/35

(52) U.S. Cl. CPC A61B 5/7221; A61B 5/0205; A61B 5/7246; A61B 5/021; A61B 5/024; A61B 5/14542; A61B 5/35

(57) ABSTRACT

A physiological information analysis device includes an obtaining unit, a determination unit, and a notification controller. The obtaining unit obtains a plurality of pieces of physiological information related to a subject. The determination unit determines effectiveness of the plurality of pieces of physiological information. The notification controller issues, when effectiveness of corresponding first physiological information and second physiological information among the plurality of pieces of physiological information matches, predetermined notification according to the effectiveness, and issues the predetermined notification according to the effectiveness of any physiological information of the first physiological information and the second physiological information when the effectiveness of the first physiological information and the effectiveness of the second physiological information do not match.

### CROSS-REFERENCE TO RELATED APPLICATIONS

This application is based on and claims priority under 35 USC 119 from Japanese Patent Application No. 2023-064123 filed on Apr. 11, 2023, the contents of which are incorporated herein by reference.

### TECHNICAL FIELD

The presently disclosed subject matter relates to a physiological information analysis device, a physiological information analysis method, and a physiological information analysis program.

### BACKGROUND ART

In a medical site such as a hospital, a patient monitor is used which monitors physiological information of a patient and outputs the physiological information of the patient or various alarms generated based on the physiological information to a healthcare worker such as a doctor. Among such patient monitors, for example, a bed-side monitor may have a function (asystole alarm function) of analyzing an electrocardiogram (ECG) of a patient and notifying a healthcare worker such as a doctor when the ECG (heart beat waveform) is flat or low in amplitude. However, an ECG waveform is also flat when electrodes of an ECG monitor are short-circuited, which is indistinguishable from the case where the waveform is flat due to asystole, and an asystole alarm may be generated.

In relation to this, JP5957379B discloses a patient monitor aimed at reducing erroneous notification of asystole. The patient monitor has a function of independently analyzing an ECG and pulses measured at the same time, and preventing notification related to asystole in a case where regular pulses are obtained even when a notification condition of asystole (for example, heart beat waveform is flat or low in amplitude) is satisfied.

However, in the patient monitor described in JP5957379B, since an influence of factors such as noise, artifacts, and respiratory variation on prevention of the notification related to asystole is not considered, the notification related to asystole may not be issued accurately.

### SUMMARY

The presently disclosed subject matter has been made in view of the above problem. Therefore, an object of the presently disclosed subject matter is to provide a physiological information analysis device, a physiological information analysis method, and a physiological information analysis program capable of accurately issuing notification related to asystole.

The above problem of the presently disclosed subject matter is solved by the following means.

A physiological information analysis device of the presently disclosed subject matter includes an obtaining unit, a determination unit, and a notification controller. The obtaining unit obtains a plurality of pieces of physiological information related to a subject. The determination unit determines effectiveness of the plurality of pieces of physiological information. The notification controller issues, when effectiveness of corresponding first physiological information and second physiological information among the plurality of pieces of physiological information matches, predetermined notification according to the effectiveness, and issues the predetermined notification according to the effectiveness of any physiological information of the first physiological information and the second physiological information when the effectiveness of the first physiological information and the effectiveness of the second physiological information do not match.

A physiological information analysis method of the presently disclosed subject matter includes: obtaining a plurality of pieces of physiological information related to a subject; determining effectiveness of the plurality of pieces of physiological information; and issuing, when effectiveness of corresponding first physiological information and second physiological information among the plurality of pieces of physiological information matches, predetermined notification according to the effectiveness, and issuing the predetermined notification according to the effectiveness of any physiological information of the first physiological information and the second physiological information when the effectiveness of the first physiological information and the effectiveness of the second physiological information do not match.

A non-transitory computer readable storage medium storing a physiological information analysis program of the presently disclosed subject matter causes a computer to execute processing including: obtaining a plurality of pieces of physiological information related to a subject; determining effectiveness of the plurality of pieces of physiological information; and issuing, when effectiveness of corresponding first physiological information and second physiological information among the plurality of pieces of physiological information matches, predetermined notification according to the effectiveness, and issuing the predetermined notification according to the effectiveness of any physiological information of the first physiological information and the second physiological information when the effectiveness of the first physiological information and the effectiveness of the second physiological information do not match.

According to the presently disclosed subject matter, since the physiological information analysis device controls prevention of the notification related to asystole based on the effectiveness of the plurality of pieces of physiological information, the notification related to asystole can be accurately issued.

### BRIEF DESCRIPTION OF DRAWINGS

FIG. 1 is a block diagram illustrating a schematic configuration of a physiological information analysis device according to a first embodiment.

FIG. 2 is a block diagram illustrating an example of a schematic configuration of an ECG processor illustrated in FIG. 1.

FIG. 3 is a block diagram illustrating a schematic configuration of a PPG processor illustrated in FIG. 1.

FIG. 4 is a flowchart illustrating an outline of a processing procedure of a physiological information analysis method performed by the physiological information analysis device of the first embodiment.

FIG. 5 is a schematic diagram illustrating an example of a correspondence relationship between a heart beat signal and a pulse signal in a case where effectiveness of the heart beat signal and effectiveness of the pulse signal match in the first embodiment.

FIG. 6 is a schematic diagram illustrating an example of a correspondence relationship between the heart beat signal and the pulse signal in a case where the effectiveness of the heart beat signal and the effectiveness of the pulse signal do not match in the first embodiment.

FIG. 7 is a schematic diagram illustrating another example of the correspondence relationship between the heart beat signal and the pulse signal in the case where the effectiveness of the heart beat signal and the effectiveness of the pulse signal do not match in the first embodiment.

FIG. 8 is a schematic diagram illustrating log information of an irregular pulse history.

FIG. 9 is a schematic diagram illustrating log information of a long-time waveform.

FIG. 10 is a subroutine flowchart illustrating a procedure of processing of determining the effectiveness of the heart beat signal (step S103) in the first embodiment.

FIG. 11 is a subroutine flowchart illustrating a procedure of processing of determining the effectiveness of the pulse signal (step S103) in the first embodiment.

FIG. 12 is a block diagram illustrating a schematic configuration of a physiological information analysis device according to a modification of the first embodiment.

FIG. 13 is a block diagram illustrating a schematic configuration of a blood pressure processor illustrated in FIG. 12.

FIG. 14 is a subroutine flowchart illustrating a procedure of processing of determining effectiveness of a pulse signal (step S103) in a second embodiment.

FIG. 15 is a subroutine flowchart illustrating a procedure of processing of determining effectiveness of a pulse signal (step S103) in a third embodiment.

FIG. 16 is a schematic diagram illustrating an example of a correspondence relationship between a heart beat signal and a pulse signal in a case where effectiveness of the heart beat signal and effectiveness of the pulse signal do not match in the third embodiment.

FIG. 17 is a schematic diagram illustrating a case where P waves appear in the heart beat signal.

FIG. 18 is a schematic diagram illustrating a case where a high-amplitude waveform appears in the heart beat signal.

FIG. 19 is a schematic diagram illustrating an example of a correspondence relationship between a heart beat signal and a blood pressure signal in a case where effectiveness of the heart beat signal and effectiveness of a blood pressure signal match in a fifth embodiment.

FIG. 20 is a schematic diagram illustrating an example of a correspondence relationship between the heart beat signal and the blood pressure signal in a case where the effectiveness of the heart beat signal and the effectiveness of the blood pressure signal do not match.

### DESCRIPTION OF EMBODIMENTS

Hereinafter, a physiological information analysis device, a physiological information analysis method, and a physiological information analysis program according to embodiments of the presently disclosed subject matter will be described in detail with reference to the drawings. In the drawings, the same members are denoted by the same reference numerals. Dimensional ratios in the drawings are exaggerated for convenience of the description, and may be different from actual ratios.

### First Embodiment

### Configuration of Physiological Information Analysis Device

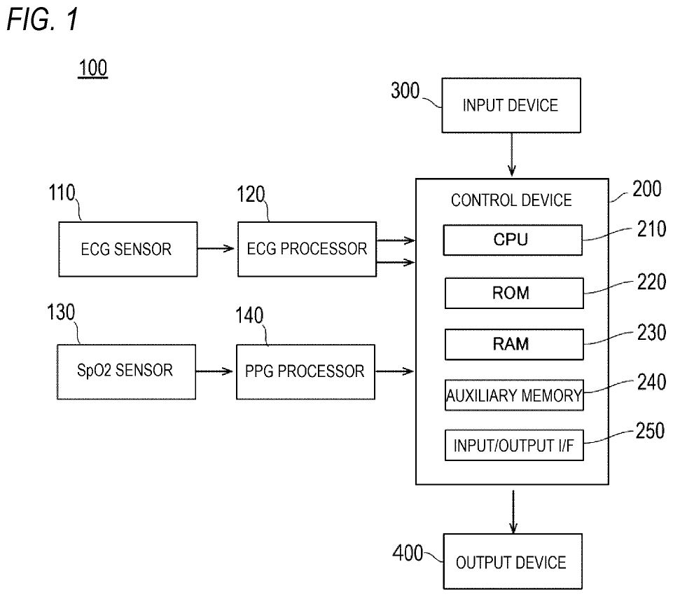

FIG. 1 is a block diagram illustrating a schematic configuration of a physiological information analysis device 100 of the present embodiment. As illustrated in the figure, the physiological information analysis device 100 can include an ECG sensor 110, an ECG processor 120, a SpO2 sensor 130, a PPG processor 140, a control device 200, an input device 300, and an output device 400. The ECG processor 120, the PPG processor 140, the input device 300, and the output device 400 are each connected to the control device 200 and controlled by the control device 200.

The ECG sensor 110 obtains physiological electrical signals from a heart through an extremity electrode portion and a chest electrode portion (both not illustrated), and transmits the signals to the ECG processor 120, the extremity electrode portion can include a plurality of electrodes for extremities, and the chest electrode portion can include a plurality of electrodes for a chest. The electrodes for extremities are attached to predetermined positions of extremities of a patient (subject), and the electrodes for chest are attached to predetermined positions of a chest of the patient.

The ECG processor 120 processes the physiological electrical signals from the ECG sensor 110 to extract a heart beat of the patient.

The SpO2 sensor 130 is a sensor that can include a light emitter for emitting light in a wavelength band absorbed by hemoglobin and a light detecting element for detecting the light from the light emitter, and that is attached to a fingertip, an ear, or the like of the patient to detect a photoplethysmography.

The PPG processor 140 processes electrical signals of the photoplethysmography from the SpO2 sensor 130 to extract pulses of the patient.

The control device 200 obtains a heart beat signal and a pulse signal as a plurality of pieces of physiological information of the patient, and monitors the information. Further, the control device 200 outputs physiological information of the patient and various alarms generated based on the physiological information to a healthcare worker such as a doctor. The physiological information analysis device 100 of the present embodiment is, for example, a bed-side monitor installed in a medical facility such as a hospital, and has a function (referred to as an “asystole alarm function”) of determining asystole of a patient based on the heart beat signal and the pulse signal and notifying a healthcare worker of the asystole. The control device 200 constitutes a computer.

The input device 300 can include, for example, a touch panel, various keys, switches, and the like, and receives instructions from a user who is a healthcare worker, various settings, information related to a patient, and the like. The instructions from the user include, for example, monitoring start/end instructions, and the various settings include, for example, mode setting, alarm setting, and the like. The information related to the patient includes sex, age, past medical history (for example, information related to cardiac disease), and the like of the patient.

The output device 400 outputs data such as an analysis result of the physiological information and an alarm. Here, the output of the output device 400 includes, for example, display of data on a display, print output to a printer, and audio output to a speaker. The output device 400 can include a display, a printer, a speaker, and the like.

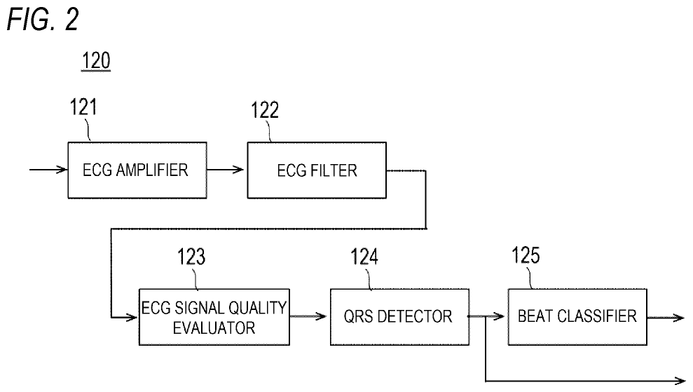

FIG. 2 is a block diagram illustrating a schematic configuration of the ECG processor 120 illustrated in FIG. 1. As illustrated in the figure, the ECG processor 120 can include an ECG amplifier 121, an ECG filter 122, an ECG signal quality evaluator 123, a QRS detector 124, and a beat classifier 125.

The ECG amplifier 121 amplifies a physiological signal received from the ECG sensor 110 (electrodes) attached to a patient to generate an ECG signal, and outputs the ECG signal to the ECG filter 122. The ECG signal indicates an action potential generated due to excitation of cardiac muscle of a patient, and includes a plurality of heart beat waveforms continuously generated on a timeline. The heart beat waveform is a waveform indicating a heart beat, that is, a pulsation of a heart.

The ECG filter 122 performs predetermined filtering processing on the ECG signal to remove a noise component contained in the ECG signal. The ECG filter 122 may include, for example, a low-pass filter or a band-pass filter.

The ECG signal quality evaluator 123 evaluates a quality of the ECG signal which has been filtered by the ECG filter 122. When the quality of the ECG signal is significantly low, a CPU 210 may control to discard the obtained data and display a warning message.

The QRS detector 124 detects beats (QRS) from an ECG signal, and generates and outputs a heart beat signal. Specifically, for example, the QRS detector 124 detects, as the beats, a portion having an amplitude equal to or greater than a predetermined amplitude in the ECG signal which has been filtered by the ECG filter 122, converts the beats into a digital signal, and outputs the digital signal as the heart beat signal to the physiological information analysis device 100.

The beat classifier 125 analyzes the beats detected by the QRS detector 124 and classifies types of the beats. The beat classifier 125 classifies the types of the beats using, for example, pattern matching. The types of the beats include a plurality of types including a normal beat (N) and an irregular beat, for example, a premature ventricular contraction beat (V).

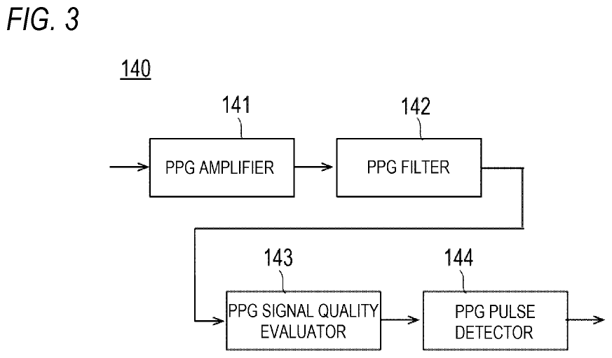

FIG. 3 is a block diagram illustrating a schematic configuration of the PPG processor 140 illustrated in FIG. 1. As illustrated in the figure, the PPG processor 140 can include a PPG amplifier 141, a PPG filter 142, a PPG signal quality evaluator 143, and a PPG pulse detector 144.

The PPG amplifier 141 receives an electrical signal of a photoplethysmography (PPG), amplifies the electric signal to a voltage signal of an appropriate magnitude, and outputs the voltage signal to the PPG filter 142.

The PPG filter 142 performs predetermined filtering processing on the voltage signal (hereinafter referred to as a “PPG signal”) of the PPG to remove a noise component contained in the PPG signal. The PPG filter 142 may include, for example, a low-pass filter or a band-pass filter.

The PPG signal quality evaluator 143 evaluates a quality of the PPG signal which has been filtered by the PPG filter 142. When the quality of the PPG signal is significantly low, the CPU 210 may control to discard the obtained data and display a warning message.

The PPG pulse detector 144 generates a pulse signal from the PPG signal and outputs the pulse signal. Specifically, the PPG pulse detector 144 converts the PPG signal which has been filtered by the PPG filter 142 into a digital signal to generate a pulse signal, and outputs the pulse signal to the control device 200.

### Hardware Configuration of Control Device 200

The control device 200 can include a central processing unit (CPU) 210, a read only memory (ROM) 220, and a random access memory (RAM) 230, an auxiliary memory 240, and an input/output I/F 250. The auxiliary memory 240 can include, for example, a solid state drive (SSD) or a hard disk drive (HDD). The CPU 210, the ROM 220, the RAM 230, and the auxiliary memory 240 constitute a computer.

The CPU 210 implements various functions by loading a physiological information analysis program stored in advance in the ROM 220 or the auxiliary memory 240 into the RAM 230 and executing the program.

The ROM 220 is a nonvolatile memory. The ROM 220 stores various parameters and the like necessary for processing in the CPU 210.

The RAM 230 is a volatile memory and temporarily stores determination results of the CPU 210 and various data.

The auxiliary memory 240 stores programs such as an operating system (OS) and the physiological information analysis program, results of processing in the CPU 210, and the like, as one of a non-transitory computer readable storage medium. In addition, the auxiliary memory 240 stores information (for example, a QRS shape or the like) related to a predetermined standard heart beat waveform (hereinafter, referred to as a “standard waveform”) and information (for example, a QRS shape or the like) related to a normal beat for each patient. Further, in the present embodiment, the auxiliary memory 240 stores a reference template generated based on information (heart beat signal) related to the heart beat as first physiological information and information (pulse signal) related to the pulse as second physiological information, and the information related to the heart beat and the information related to the pulse are obtained during monitoring. Details of the reference template will be described later.

The input/output I/F 250 is an input/output interface that transmits and receives data to and from the ECG processor 120, the PPG processor 140, the input device 300, and the output device 400 according to a predetermined communication protocol.

The CPU 210 implements various functions by executing the physiological information analysis program. For example, the CPU 210 integrally controls the ECG sensor 110, the ECG processor 120, the Sp02 sensor 130, the PPG processor 140, the control device 200, the input device 300, and the output device 400.

Specifically, when the monitoring is started, the CPU 210 constantly obtains the heart beat signal and the pulse signal of the patient through the input/output I/F 250, analyzes the signals, and outputs analysis results and various alarms to the output device 400. The output device 400 displays the analysis results and the various alarms on a display.

Since a basic configuration for analyzing and displaying the physiological information of the patient in the physiological information analysis device 100 is the same as or similar to that of the patient monitor in the related art, a detailed description thereof will be omitted, and the asystole alarm function of the present embodiment will be mainly described below. In the asystole alarm function, the CPU 210, the RAM 230, and the input/output I/F 250 constitute an obtaining unit. Further, the CPU 210 constitutes a determination unit, a notification controller, a mean pulse calculator, and a log output unit.

### Physiological Information Analysis Method

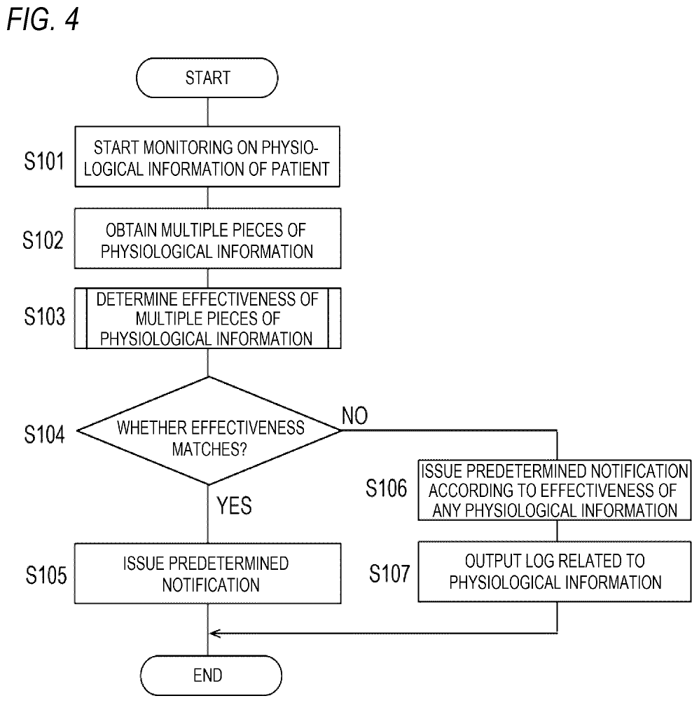

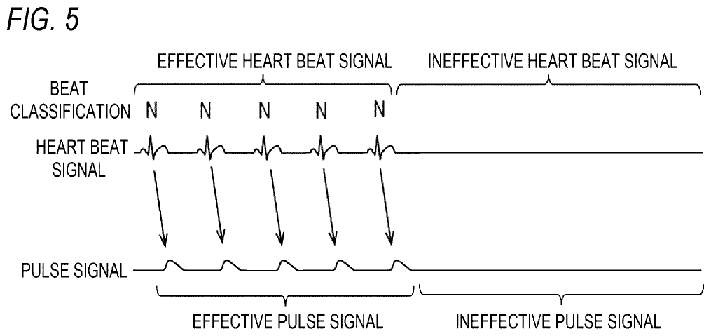

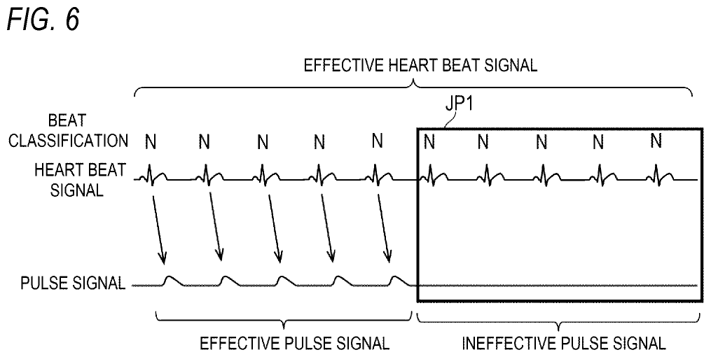

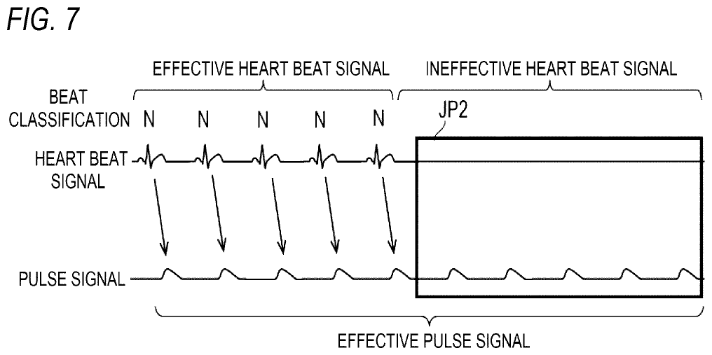

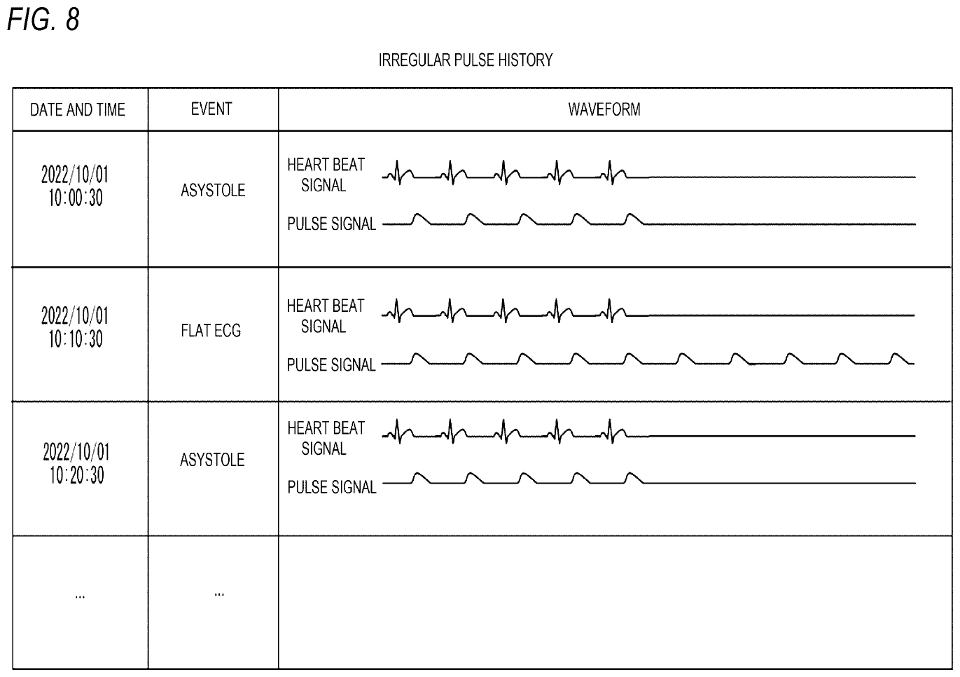

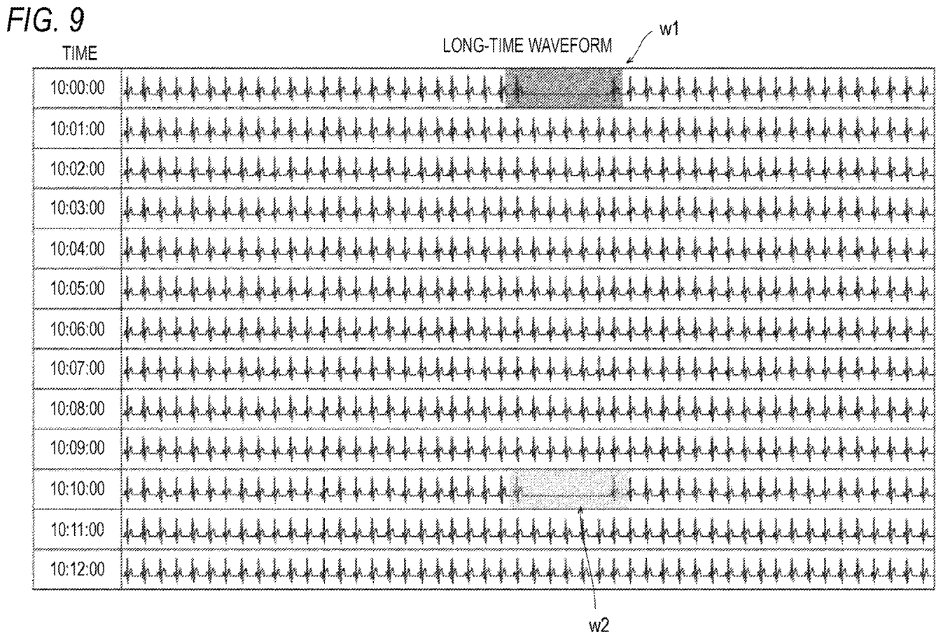

Hereinafter, an outline of a processing procedure of the physiological information analysis method performed by the physiological information analysis device 100 will be described with reference to FIGS. 4 to 7. FIG. 4 is a flowchart illustrating the outline of the processing procedure of the physiological information analysis method performed by the physiological information analysis device 100 of the first embodiment. The processing of the flowchart illustrated in the figure is implemented by the CPU 210 executing the physiological information analysis program. FIG. 5 is a schematic diagram illustrating an example of a correspondence relationship between the heart beat signal and the pulse signal in a case where effectiveness of the heart beat signal and effectiveness of the pulse signal match. FIG. 6 is a schematic diagram illustrating an example of the correspondence relationship between the heart beat signal and the pulse signal in a case where the effectiveness of the heart beat signal and the effectiveness of the pulse signal do not match, and FIG. 7 is a schematic diagram illustrating another example of the correspondence relationship between the heart beat signal and the pulse signal in the case where the effectiveness of the heart beat signal and the effectiveness of the pulse signal do not match. Further, FIG. 8 is a schematic diagram illustrating log information of an irregular pulse history, and FIG. 9 is a schematic diagram illustrating log information of a long-time waveform.

As illustrated in FIG. 4, the CPU 210 starts monitoring on physiological information of a patient (step S101). Upon receiving a monitoring start instruction from a user through the input device 300, the CPU 210 starts monitoring on the physiological information (heart beat signal and pulse signal) of the patient.

The obtaining unit obtains a plurality of pieces of physiological information (step S102). The obtaining unit constantly obtains the heart beat signal and the pulse signal as the physiological information of the patient during the monitoring. For example, the obtaining unit sequentially stores, into the RAM 230, data including at least eight past beats among the obtained heart beat signal and pulse signal.

As illustrated in FIG. 5, since the pulse is generated due to the heart beat, and the QRS of the heart beat signal and the pulse of the pulse signal have a one-to-one correspondence (in the figure, the correspondence relationship is illustrated by arrows) during normal times. Therefore, when the QRS of the heart beat signal and the pulse of the pulse signal have a one-to-one correspondence and a delay time from the QRS to the pulse is constant during normal times, the QRS and the pulse occur by a common heart beat, and is guaranteed to be a heart beat and a pulse corresponding to the heart beat of the same patient (for details, refer to Xianliang He et al., Method to Determine the Applicability of Multi-Parameter Fusion Analysis, 2019, Computing in Cardiology 2019, and 46 Vol).

The mean pulse calculator calculates a mean amplitude and a mean interval of the pulses by using the pulse signals obtained during normal times, as preprocessing of processing of a next step (step S103), and stores the mean amplitude and the mean interval into the RAM 230. Further, the mean pulse calculator calculates the mean amplitude and the mean interval of the pulses as appropriate, and updates the data in the RAM 230. More specifically, the mean pulse calculator calculates and updates, for each beat (for example, every time a new QRS is detected), a mean amplitude and a mean interval of the pulses corresponding to N past beats stored in the RAM 230. Alternatively, regarding the amplitude and the interval of the pulse, the mean pulse calculator can also calculate (N−1)/N times a mean value+1/N times a latest value for each beat, and update the mean amplitude and the mean interval of the pulse. Here, N is any natural number, and is preferably set to 8, for example, and a natural number such as 16 or 32 may also be applied.

Next, the determination unit determines effectiveness of the plurality of pieces of physiological information (step S103). The determination unit determines the effectiveness of the plurality of pieces of physiological information according to a procedure of a predetermined determination processing. FIGS. 5 to 7 illustrate determination results of the effectiveness regarding the heart beat signal and the pulse signal. A specific procedure of the determination processing will be described later.

Next, the determination unit determines whether the effectiveness of the plurality of pieces of physiological information matches (step S104). For example, when both the heart beat signal and the pulse signal are effective, or when both the heart beat signal and the pulse signal are ineffective, the determination unit determines that the effectiveness of the heart beat signal and the effectiveness of the pulse signal match. FIG. 5 illustrates a case where both the heart beat signal and the pulse signal change from effective to ineffective. In this case, it is considered that the heart beat of the patient stops and the patient gets into an asystole state, and thus the pulse also stops. The asystole of the patient is a state to be notified to the healthcare worker.

A portion JP1 in a square of FIG. 6 represents a determination target period of the determination unit. When the heart beat signal is effective and the pulse signal is ineffective, the determination unit determines that the effectiveness of the heart beat signal and the effectiveness of the pulse signal do not match. The figure illustrates a case where the heart beat signal is maintained effective and the pulse signal is changed from effective to ineffective. In this case, the heart beat signal is maintained for some reason, but the pulse signal is stopped. The determination unit may erroneously recognize a high-amplitude wave as the QRS, and determine that the heart beat signal is effective. This case is a state to be notified to the healthcare worker.

Meanwhile, in general, such a situation does not occur in a case where the ECG sensor 110 and the SpO2 sensor 130 are attached to the same patient and perform correct measurements, and the patient is not in an asystole state. Accordingly, the SpO2 sensor 130 may be detached from the patient.

A portion JP2 in a square of FIG. 7 represents a determination target period of the determination unit. When the heart beat signal is ineffective and the pulse signal is effective, the determination unit also determines that the effectiveness of the heart beat signal and the effectiveness of the pulse signal do not match. The figure illustrates a case where the heart beat signal changes from effective to ineffective and the pulse signal is maintained effective. In this case, the heart beat signal is stopped for some reason, but the pulse signal is maintained. Accordingly, it is considered that the heart beat of the patient is not stopped, and it is not necessary to notify the healthcare worker of asystole.

Meanwhile, in general, such a situation does not occur in a case where the ECG sensor 110 and the Sp02 sensor 130 are normally attached to the same patient and perform correct measurements. Accordingly, the ECG sensor 110 may be detached from the patient or short-circuited.

When the effectiveness of the heart beat signal and the effectiveness of the pulse signal match (step S104: YES), the notification controller issues predetermined notification according to the effectiveness (step S105). In the present embodiment, the predetermined notification is notification related to asystole of a patient (for example, an asystole alarm or a ventricular fibrillation alarm). In the present embodiment, the notification controller issues predetermined notification as illustrated in Table 1 below according to the effectiveness of the heart beat signal and the effectiveness of the pulse signal.

TABLE 1

| | Heart beat | | Presence or absence of |
|---|---|---|---|
| Effectiveness | signal | Pulse signal | predetermined notification |
| Effectiveness | Effective | Effective | No notification |
| matches | Ineffective | Ineffective | Notification |
| Effectiveness | Effective | Ineffective | Notification |
| does not match | Ineffective | Effective | No notification |

For example, when both the heart beat signal and the pulse signal are effective, the notification controller does not issue the notification because it is estimated that the patient is not in an asystole state. That is, when the heart beat signal is effective, a condition for issuing the predetermined notification (for example, asystole alarm condition) is not satisfied, and when the pulse signal is effective, the predetermined notification is prevented (a condition for preventing the predetermined notification is satisfied). A predetermined time may be, for example, 5 seconds.

On the other hand, when both the heart beat signal and the pulse signal are ineffective, it is estimated that the patient is in an asystole state, and thus the notification controller issues notification related to asystole as the predetermined notification. That is, when the heart beat signal is ineffective, a condition for issuing the notification related to asystole is satisfied, but since the pulse signal is ineffective, the condition for preventing the predetermined notification is not satisfied.

On the other hand, when the effectiveness of the heart beat signal and the effectiveness of the pulse signal do not match (step S104: NO), the notification controller issues the predetermined notification according to the effectiveness of any physiological information in the heart beat signal and the pulse signal (step S106). In the present embodiment, for example, the notification controller may be determined in advance to issue the predetermined notification according to the effectiveness of the pulse signal. For example, when the heart beat signal is effective and the pulse signal is ineffective, the notification controller does not prevent the predetermined notification (issues the predetermined notification) based on the fact that the pulse signal is ineffective.

When the heart beat signal is ineffective and the pulse signal is effective, the notification controller prevents the predetermined notification (does not issue the predetermined notification) based on the fact that the pulse signal is effective. That is, as a result of comparing, by the determination unit, the pulse signal when the heart beat signal is effective with the pulse signal when the heart beat signal is ineffective, as long as the pulse signal is maintained effective and does not change, the notification controller does not issue the notification related to asystole. The condition for issuing the predetermined notification is satisfied when the heart beat signal is ineffective, but the condition for preventing the predetermined notification is also simultaneously satisfied when the pulse signal is effective, and therefore the notification controller controls to prevent the predetermined notification by giving priority to the effectiveness of the pulse signal.

Next, the log output unit outputs log information (history) related to the physiological information (step S107). More specifically, the log output unit outputs the log information related to the physiological information when the effectiveness of the heart beat signal and the effectiveness of the pulse signal do not match. This is because, in a case where the heart beat signal is ineffective (that is, the ECG is flat or low in amplitude) and the condition for issuing the predetermined notification is satisfied, it is possible for the healthcare worker to check later by leaving in the data log that the predetermined notification is prevented because the pulse signal is effective.

As illustrated in FIG. 8, the log output unit can output (display/print) an irregular pulse history or store the irregular pulse history into the auxiliary memory 240, as the log information. The irregular pulse history includes, for example, an event, a date and time when the event occurs, and a waveform related to the event. In addition, the event may include, for example, asystole, flat ECG, atrial fibrillation (AF), ventricular fibrillation (VF), and the like.

As illustrated in FIG. 9, the log output unit can output (display/print) a long-time waveform or store the long-time waveform into the auxiliary memory 240, as the log information. The log output unit can output the long-time waveform with an identifier such as a color or a mark attached to an alarm generator. This makes it easier for the healthcare worker to distinguish the waveform when the alarm is generated. FIG. 9 illustrates a case where a waveform w1 when the asystole alarm is notified and a waveform w2 when the asystole alarm is prevented based on the pulse signal are displayed with different colors (for example, red and green).

In this way, in the processing of the flowchart illustrated in FIG. 4, the control device 200 obtains the heart beat signal and the pulse signal of the patient, and determines the effectiveness of the heart beat signal and the effectiveness of the pulse signal. Then, as a result of the determination of the effectiveness, when the effectiveness of the heart beat signal and the effectiveness of the pulse signal match, the control device 200 issues the predetermined notification according to the effectiveness, and when the effectiveness of the heart beat signal and the effectiveness of the pulse signal do not match, the control device 200 issues the predetermined notification according to the effectiveness of the pulse signal.

In the example described above, a case has been described in which it is determined in advance that the predetermined notification is issued according to the effectiveness of the pulse signal when the effectiveness of the heart beat signal and the effectiveness of the pulse signal do not match. However, the present embodiment is not limited to such a case, and it may be determined in advance that the predetermined notification is issued according to the effectiveness of the heart beat signal when the effectiveness of the heart beat signal and the effectiveness of the pulse signal do not match.

### Determination of Effectiveness of Heart Beat Signal (Step S103)

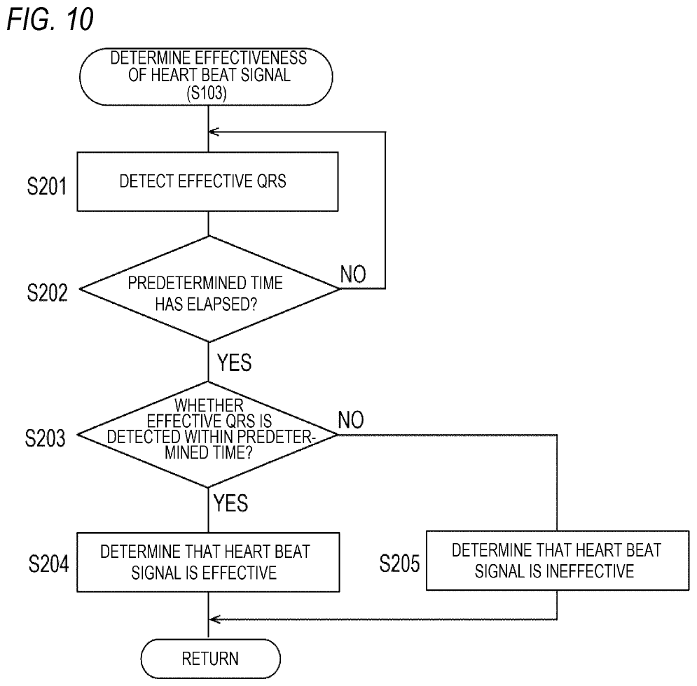

FIG. 10 is a subroutine flowchart illustrating a procedure of the processing of determining the effectiveness of the heart beat signal (step S103). The processing of the flowchart illustrated in the figure is implemented by the CPU 210 executing the physiological information analysis program.

First, the determination unit detects an effective QRS in the heart beat signal (step S201). The determination unit determines that the effective QRS is detected, for example, when a QRS shape of the heart beat signal matches a predetermined QRS shape according to a predetermined ECG analysis, for example, a template matching method and the QRS shape is determined to be Normal (N) as a result of beat classification of the beat classifier 125. The predetermined QRS shape is the same as a QRS shape of a standard waveform or a QRS shape of a normal beat for each patient. For example, the QRS shape of the normal beat of a patient can be obtained by averaging the QRS shape of the heart beat signal measured during normal times or learning the normal QRS shape for each patient in advance. Accordingly, the normal beat may vary to some extent depending on a disease or a state of a patient among patients. For example, in patients with bundle branch block or patients using pacemakers, the normal beat may have a QRS shape that is wider and looks like premature ventricular contraction. As described above, by determining whether the QRS shape of the heart beat signal matches a predetermined QRS shape, the effective QRS of patients in various states can be detected.

Next, the determination unit determines whether a predetermined time has elapsed (step S202). When the predetermined time has not elapsed (step S202: NO), the determination unit returns to the processing of step S201 and continues to detect the QRS until the predetermined time elapses.

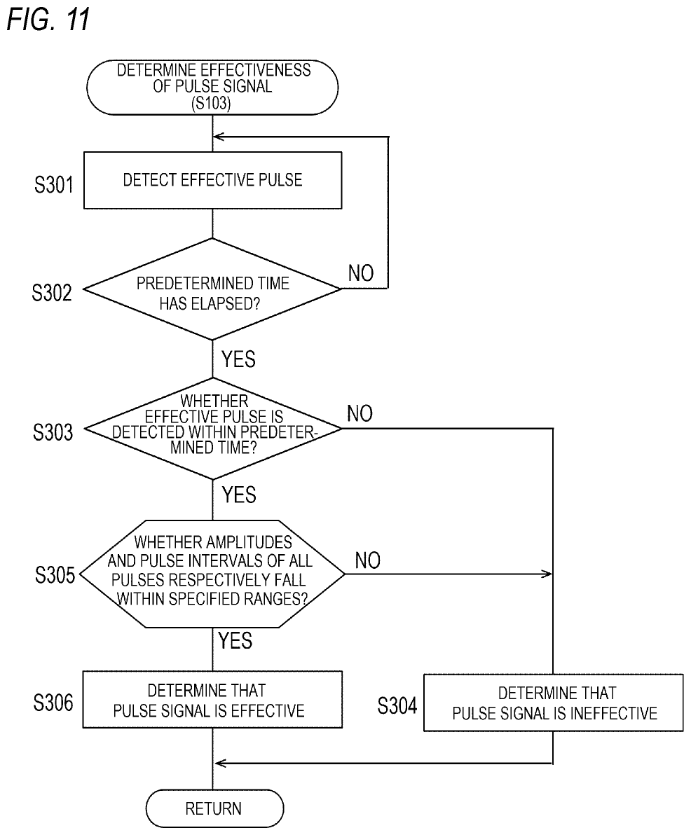

On the other hand, when the predetermined time has elapsed (step S202: YES), the determination unit determines whether the effective QRS is detected within a predetermined time (step S203). When the effective QRS is detected in the predetermined time (step S203: YES), the determination unit determines that the heart beat signal is effective (step S204) and ends the processing (return). On the other hand, when no effective QRS is detected in the heart beat signal for a predetermined time (step S203: NO), the determination unit determines that the heart beat signal is ineffective (step S205), and ends the processing (return).

[Determination of Effectiveness of Pulse Signal (Step S103)] FIG. 11 is a subroutine flowchart illustrating the procedure of the processing of determining the effectiveness of the pulse signal (step S103). The processing of the flowchart illustrated in the figure is implemented by the CPU 210 executing the physiological information analysis program.

First, the determination unit detects an effective pulse in the pulse signal (step S301). For example, the determination unit determines that an effective pulse is detected when an amplitude is equal to or greater than a predetermined threshold.

Next, it is determined whether a predetermined time has elapsed (step S302). When the predetermined time has not elapsed (step S302: NO), the determination unit returns to the processing of step S301 and continues to detect the pulse until the predetermined time elapses. The predetermined time may be, for example, 5 seconds.

On the other hand, when the predetermined time has elapsed (step S302: YES), the determination unit determines whether the effective pulse is detected within the predetermined time (step S303). When no effective pulse is detected within the predetermined time (step S303: NO), the determination unit determines that the pulse signal is ineffective (step S304). On the other hand, when the effective pulse is detected within the predetermined time (step S303: YES), the determination unit determines whether amplitudes and pulse intervals of all the pulses respectively fall within specified ranges (step S305). When the amplitude and the pulse interval respectively fall within the specified ranges for all the pulses (step S305: YES), the determination unit determines that the pulse signal is effective (step S306). On the other hand, when the amplitude or the pulse interval does not fall within the specified range for at least one of the pulses (step S305: NO), it is determined that the pulse signal is ineffective (step S304). The specified ranges of the amplitude and the interval may be a mean value±an allowable error of the amplitude and a mean value±an allowable error of the interval, respectively. The allowable error may be, for example, a standard deviation.

As described above, in the present embodiment, the effectiveness of the pulse is determined based on continuity obtained from the mean amplitude and the mean interval during normal times. Specifically, the mean amplitude and the mean interval of the pulses are calculated and updated as appropriate during monitoring. For example, the determination unit determines that the effective pulses continue if the amplitudes and the intervals of all the pulses within the predetermined time respectively fall within the specified ranges each of which is the mean value±allowable error (for example, standard deviation), and determines that the effective pulses do not continue if the amplitudes and the intervals do not fall within the specified ranges respectively. Accordingly, it can be confirmed that the detected pulse is a pulse caused by the heart beat of the patient and is an effective pulse.

### Modification

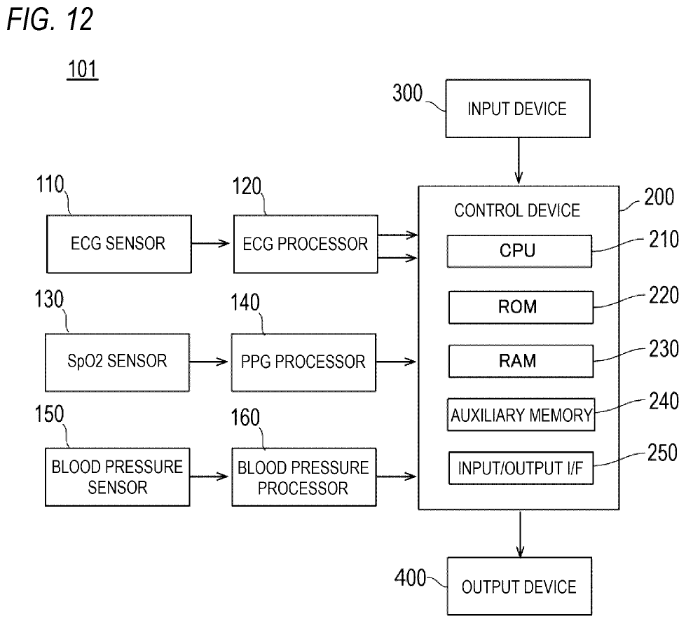

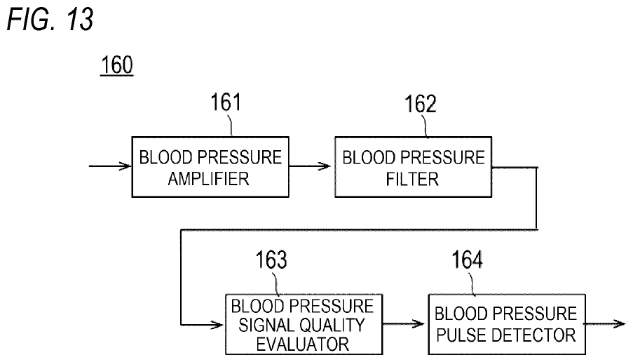

FIG. 12 is a block diagram illustrating a schematic configuration of a physiological information analysis device 101 according to a modification of the first embodiment, and FIG. 13 is a block diagram illustrating a schematic configuration of a blood pressure processor 160 illustrated in FIG. 12.

In the above example, a case has been described in which the electrical signal of the PPG from the SpO2 sensor 130 is processed to extract a pulse of a patient, but instead of the SpO2 sensor 130, an electrical signal of a pressure from a blood pressure sensor 150 may be processed to extract a pulse of a patient, as illustrated in FIG. 12. The blood pressure sensor 150 may be, for example, an invasive blood pressure sensor (transducer) or a non invasive blood pressure sensor (cuff+pressure sensor). Hereinafter, a case will be described in which the blood pressure sensor 150 is an invasive blood pressure sensor.

As illustrated in FIG. 13, the blood pressure processor 160 can include a blood pressure amplifier 161, a blood pressure filter 162, a blood pressure signal quality evaluator 163, and a blood pressure pulse detector 164.

The blood pressure amplifier 161 receives an electrical signal of a pressure detected by a transducer, amplifies the electrical signal to a voltage signal of an appropriate magnitude, and outputs the voltage signal to the blood pressure filter 162.

The blood pressure filter 162 performs predetermined filtering processing on the amplified voltage signal to remove a noise component contained in the voltage signal. The blood pressure filter 162 may include, for example, a low-pass filter or a band-pass filter.

The blood pressure signal quality evaluator 163 evaluates a quality of the voltage signal which has been filtered by the blood pressure filter 162. When the quality of the voltage signal is significantly low, the CPU 210 may control to discard the obtained data and display a warning message.

The blood pressure pulse detector 164 detects the pulse from the voltage signal which has been filtered by the blood pressure filter 162, and generates and outputs the pulse signal. Specifically, the blood pressure pulse detector 164 converts the voltage signal which has been filtered by the blood pressure filter 162 into a digital signal to generate the pulse signal, and outputs the pulse signal to the control device 200.

The control device 200 determines, according to the physiological information analysis method described above, the effectiveness of the heart beat signal and the effectiveness of the pulse signal of the patient using the pulse signal extracted by the blood pressure processor 160, and issues notification related to asystole according to the determination result. The specific processing procedure is the same as that in the case of using the pulse signal obtained based on the PPG, and a detailed description thereof will be omitted.

According to the physiological information analysis device 100 of the present embodiment described above, the following effects can be achieved.

Since the physiological information analysis device 100 controls prevention of the notification related to asystole based on the effectiveness of a plurality of pieces of physiological information, the notification related to asystole can be accurately issued. In particular, in the present embodiment, it is possible to more accurately determine whether to issue the notification related to asystole by analyzing the ECG, which is related to the heart beat, and the pulse in association with each other.

For example, in a case where a pulse-shaped regular signal is accidentally mixed into the pulse signal due to noise or artifact, a condition of the effective pulse signal is not satisfied, and the notification related to asystole is not prevented. Here, the condition of the effective pulse signal is that the effective pulse is detected within a predetermined time, and the amplitudes and the pulse intervals of all the pulses respectively fall within specified ranges. Accordingly, excessive prevention of the notification related to asystole can be prevented.

Further, in a case where the ECG and the pulse are obtained from different patients due to the fact that the ECG sensor 110 and the SpO2 sensor 130 (blood pressure sensor 150) are erroneously attached to different patients, the condition of the effective pulse signal is also not satisfied, and the notification related to asystole is not prevented. Accordingly, excessive prevention of an asystole alarm can be prevented.

In the present embodiment, the effectiveness of the pulse signal is determined based on whether the interval of the pulse is constant, that is, whether the pulse interval falls within a specified range, rather than regularity of the pulse. Accordingly, even when the interval of the pulse gradually changes as in respiratory variation, the notification related to asystole can be issued.

### Second Embodiment

In the first embodiment, a case has been described in which the effectiveness of the pulse signal is determined based on the continuity obtained from the mean amplitude and the mean interval during normal times. In a second embodiment, a case will be described in which the effectiveness of the pulse signal is determined based on a result of comparing a shape of a pulse in the pulse signal with a shape of a pulse in a reference template generated in advance. In order to avoid redundant description, detailed description of the same configuration as that of the first embodiment will be omitted.

A physiological information analysis method performed by the physiological information analysis device 100 of the present embodiment is the same as the physiological information analysis method performed by the physiological information analysis device 100 of the first embodiment except the processing of determining the effectiveness of the pulse signal (step S103 of the flowchart illustrated in FIG. 4). Therefore, in the following description, the processing of determining the effectiveness of the pulse signal and the preprocessing thereof will be described in detail, and the description of the processing common to the first embodiment will be omitted.

### Preprocessing

The CPU 210 functions as a reference pulse creation unit, and creates a reference template using the heart beat signal and the pulse signal obtained during normal times, as preprocessing of the processing of step S103 in the flowchart of FIG. 4. More specifically, the reference pulse creation unit handles the QRS of the heart beat signal and the pulse of the corresponding pulse signal as a group (hereinafter referred to as a “heart beat template group”), collects similar features from the generated heart beat template group during monitoring, and adds and averages the collected features to create a reference template related to each feature. Examples of the features include a QRS shape, a pulse shape, an amplitude extracted from the pulse, a width of the pulse, a slope of the pulse, and a delay time from the QRS to the pulse. The reference template is generated when a QRS shape of the heart beat signal matches a predetermined QRS shape according to a template matching method and the QRS shape is determined to be Normal (N) as a result of beat classification of the beat classifier 125.

As a mean waveform of the QRS, an addition average waveform is created based on a QRS peak. In addition, as a mean waveform of the pulse, an addition average waveform may also be created based on the QRS peak. Alternatively, as the mean waveform of the pulse, an addition average waveform may be created based on a pulse peak. This is because, when the mean waveform of the pulse is created based on the QRS peak, a fluctuation of a delay time from the QRS to the pulse may affect a shape of the mean waveform.

The CPU 210 functions as a measurement unit and measures a delay time from the detection of the QRS (heart beat) to the detection of the pulse. In the present embodiment, the measurement unit may be configured to measure a time from the QRS peak to the pulse peak as the delay time. When it is determined that the delay time from the QRS to the pulse is constant, it can be confirmed that the QRS and the pulse are caused by a common heart beat. The measurement unit may be configured to measure a time from a start of the QRS to a rise of the pulse as the delay time, but is preferably configured to measure a time between peaks from the viewpoint of stability of the measurement. The notification controller determines that the pulse of the pulse signal corresponds to the heart beat of the heart beat signal when the delay time is equal to or less than a predetermined threshold (second threshold), and determines that the pulse of the pulse signal does not correspond to the heart beat of the heart beat signal when the delay time exceeds the predetermined threshold.

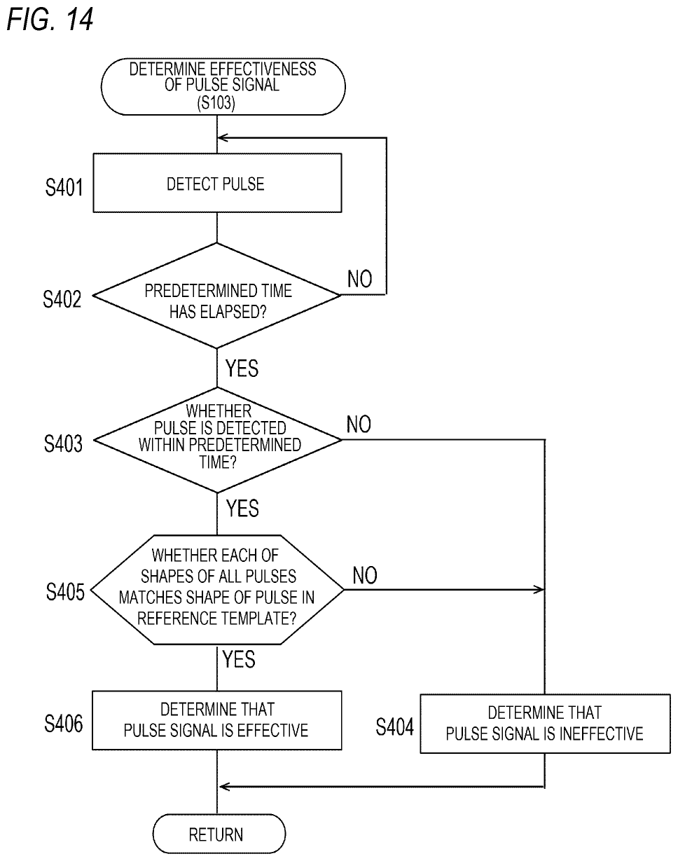

FIG. 14 is a subroutine flowchart illustrating a procedure of the processing of determining the effectiveness of the pulse signal (step S103) in the second embodiment. The processing of the subroutine flowchart illustrated in the figure is implemented by the CPU 210 executing the physiological information analysis program.

Processing of steps S401 to S404 and S406 are the same as the processing of steps S301 to S304 and S306 in the processing of determining the effectiveness of the pulse signal in the first embodiment illustrated in FIG. 11, and thus detailed description thereof will be omitted.

In step S405, it is determined whether each of shapes of all the pulses matches the shape of the pulse in the reference template. The determination unit determines whether each of the shapes of all the pulses detected within a predetermined time substantially matches the shape of the pulse in the reference template according to, for example, template matching.

The determination unit may determine whether the both pulses match according to template matching by using, for example, a determination method such as calculating a cross-correlation between a waveform of the detected pulse and a waveform of the pulse in the reference template, or calculating a difference when the waveforms of the both pulses are superimposed.

For example, the determination unit calculates a correlation coefficient for the waveform data of the both pulses, and determines that the both pulses substantially match when the correlation coefficient is equal to or greater than a predetermined first determination threshold, and determines that the both pulses do not match when the correlation coefficient is less than the predetermined first determination threshold. For example, the determination unit calculates a difference when the waveforms of the both pulses are superimposed, and determines that the both pulses substantially match when the difference is equal to or less than a predetermined second determination threshold, and determines that the both pulses do not match when the difference exceeds the predetermined second determination threshold.

When the shape of the detected pulse substantially matches the shape of the pulse in the reference template, the interval of the detected pulse may not be necessarily constant.

In a case of an AF patient, an amplitude of the pulse is not constant. Therefore, for example, the determination unit may be configured to give a margin to the first or second determination threshold (that is, loosen the determination criterion) to the AF patient based on the information related to the patient.

In addition, in a case where an irregular pulse (for example, extrasystole) occurs during a determination target period, each of shape of a part of the pulses may not match the reference template. Therefore, the determination unit may be configured to, instead of determining whether each of the shapes of all the pulses matches the shape of the pulse in the reference template, determine whether each of shapes of a predetermined number or more of pulses matches the shape of the pulse in the reference template. The determination unit determines that the pulse signal is effective when each of shapes of a predetermined number (first threshold) or more of pulses matches the shape of the pulse in the reference template during a predetermined time in which the heart beat signal is ineffective. On the other hand, the determination unit determines that the pulse signal is ineffective when each of shapes of a predetermined number or less of pulses matches the shape of the pulse in the reference template or when there is no pulse whose shape matches the shape of the pulse in the reference template. The predetermined number may be, for example, the number of pulses—1 or ¾ of the number of pulses with respect to all the pulses in a predetermined time period in which the heart beat signal is ineffective.

According to the physiological information analysis device 100 of the present embodiment described above, the following effects can be achieved in addition to the effects of the first embodiment.

For a case where an irregular heart beat such as AF occurs, the notification related to asystole can also be issued.

In addition, for a case where an irregular pulse occurs during a determination target period, the notification related to asystole can also be issued.

### Third Embodiment

In a third embodiment, a case will be described in which a timing at which the pulse is to appear if there is no asystole is estimated, and the effectiveness of the pulse signal is determined based on whether the pulse is detected at that timing. In order to avoid redundant description, detailed description of the same configuration as that of the second embodiment will be omitted.

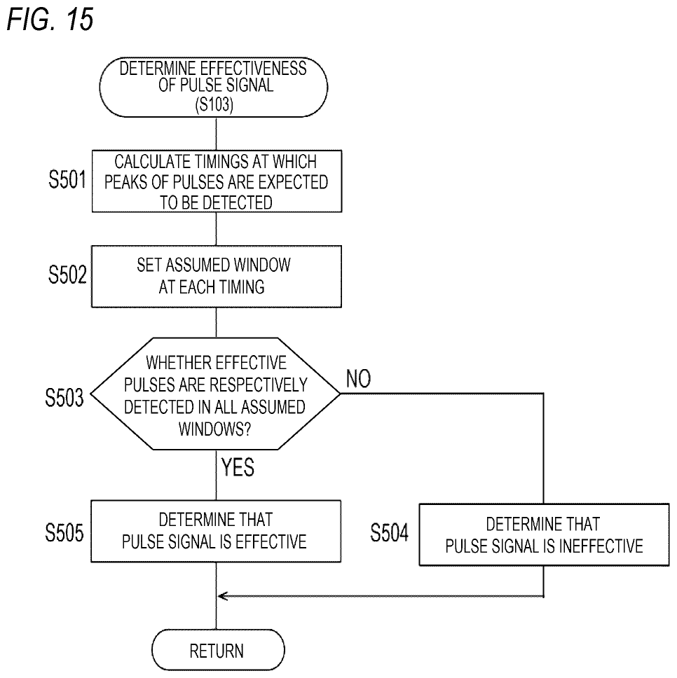

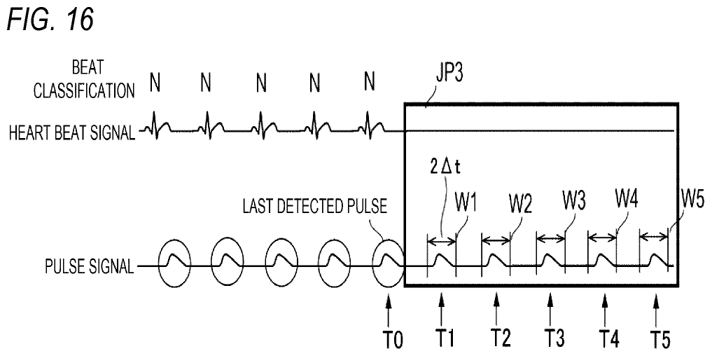

FIG. 15 is a subroutine flowchart illustrating a procedure of the processing of determining the effectiveness of the pulse signal (step S103) in the third embodiment. The processing of the subroutine flowchart illustrated in the figure is implemented by the CPU 210 executing the physiological information analysis program. FIG. 16 is a schematic diagram illustrating an example of a correspondence relationship between the heart beat signal and the pulse signal in a case where the effectiveness of the heart beat signal and the effectiveness of the pulse signal do not match in the third embodiment.

In the present embodiment, the CPU 210 functions as a pulse interval calculator and a pulse interval estimator. The pulse interval calculator calculates a mean pulse interval of the pulses during a period in which the heart beat signal is effective. Further, the pulse interval estimator estimates, based on the mean pulse interval, appearance timings of the pulses during a predetermined time at which the heart beat signal is ineffective.

As illustrated in FIG. 15, first, the determination unit calculates timings at which peaks of pulses are expected to be detected during a determination target period JP3 illustrated in FIG. 16 (step S501). The determination unit calculates, based on the heart beat template group generated during a period in which the QRS is detected, timings T=T1, T2, . . . to T5 at each of which the peak of the pulse is expected to be detected during the determination target period JP3. In the figure, the effective pulses of the heart beat template group are each indicated in a circle. The timing T can be calculated by a peak TO of a last detected pulse in the heart beat template group+a mean interval of the pulse×m (m is a natural number).

Next, the determination unit sets an assumed window at each of the timings (step S502). The determination unit sets the assumed windows W=W1, W2, . . . , W5 to T−Δt to T+Δt with respect to the timing T at which the pulse is to appear. For example, Δt may be 80 [ms].

Next, it is determined whether effective pulses are respectively detected in all the assumed windows W (step S503). The determination unit may determine whether the effective pulse is detected based on features of the pulse (for example, an amplitude, and a rise time). Alternatively, the determination unit may determine whether the effective pulse is detected, based on whether the shape of the detected pulse matches the shape of the pulse in the reference template by template matching.

According to the physiological information analysis device 100 of the present embodiment described above, the following effects can be achieved in addition to the effects of the first and second embodiments.

Since the physiological information analysis device 100 determines whether the effective pulse is detected, based on the timing at which the pulse is to appear and the features of the pulse, whether the effective pulse is detected can be determined more accurately.

### Fourth Embodiment

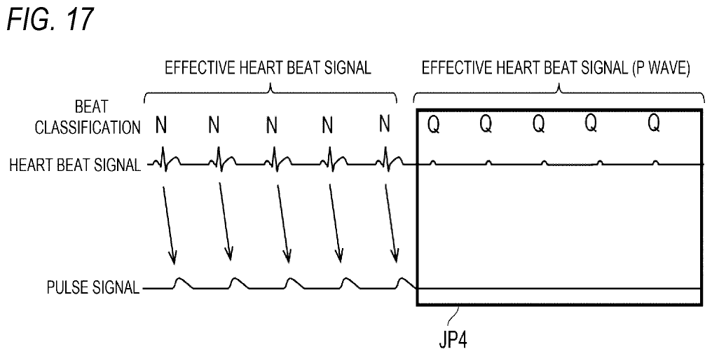

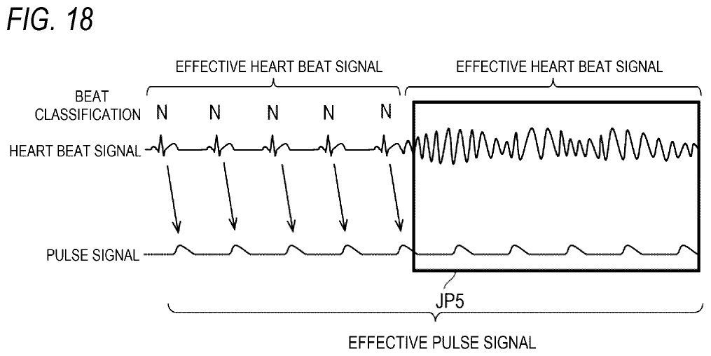

In a fourth embodiment, a case will be described in which a waveform of the heart beat signal is further analyzed when the heart beat signal is effective and the pulse signal is ineffective. FIG. 17 is a schematic diagram illustrating a case where P waves appear in the heart beat signal, and FIG. 18 is a schematic diagram illustrating a case where a high-amplitude waveform appears in the heart beat signal. In order to avoid redundant description, detailed description of the same configuration as that of the first embodiment will be omitted.

As illustrated in a determination target period JP4 in FIG. 17, for example, only P waves may continue in a case where stimulation is not transmitted from the atrium to the ventricle due to ventricular block. In such a case, the determination unit may erroneously recognize the high-amplitude P wave as the QRS, and determine that the heart beat signal is effective.

In addition, as illustrated in a determination target period JP5 in FIG. 18, in a case where a high-amplitude waveform appears in the heart beat signal, the determination unit may erroneously recognize the high-amplitude waveform as the QRS, and determine that the heart beat signal is effective.

As illustrated in Table 2 below, for example, in the case where a high-amplitude waveform appears in the heart beat signal such as a P wave or VF, the determination unit may erroneously recognize the high-amplitude waveform as the QRS. Therefore, in the present embodiment, the determination unit is configured to, when the heart beat signal is effective and the pulse signal is ineffective, further analyze the waveform of the heart beat signal and determine whether the waveform is likely to be a P wave or a VF waveform.

TABLE 2

| | | | | Presence or |
|---|---|---|---|---|
| | | | | absence of |
| | | | Pulse | predetermined |
| Effectiveness | Heart beat signal | | signal | notification |
| Effectiveness | Effective | P wave | Effective | No notification |
| matches | | VF | Effective | No notification |
| | | QRS detected | Effective | No notification |
| | Ineffective | | Ineffective | Notification |
| Effectiveness | Effective | P wave | Ineffective | Notification |
| does not match | | VF | Ineffective | Notification |
| | | QRS detected | Ineffective | Notification |
| | Ineffective | | Effective | No notification |

For example, when the waveform of the heart beat signal satisfies any one of the following (a) and (b), the determination unit determines that the waveform of the heart beat signal is likely to be the P wave.

(a) An amplitude of the waveform of the heart beat signal is extremely smaller than an amplitude of the QRS in a normal beat of a patient (hereinafter, simply referred to as “normal beat”) stored in advance in the auxiliary memory 240 (for example, less than ⅓ of the normal beat).

(b) The waveform of the heart beat signal is equal or similar to a shape of the P wave of the normal beat. For example, the determination unit may be configured to determine whether the waveform of the heart beat signal matches the P wave of the normal beat using template matching or the like. Further, when the waveform of the heart beat signal and the P wave of the normal beat have substantially the same waveform features (amplitude, width, polarity, and area), the determination unit determines that the waveform of the heart beat signal is equal or similar to the shape of the P wave of the normal beat.

Same or similarly, the determination unit determines whether the waveform of the heart beat signal is likely to be the VF waveform. For example, the determination unit determines that the waveform of the heart beat signal is likely to be the VF waveform by frequency analysis or counting the number of waveforms having a specific amplitude (see, for example, JP2020-156927A for a VF determination method). If the waveform of the heart beat signal is the VF waveform, it is considered that the pulse does not appear because a pump function of a heart of the patient is lost the same as or similarly to the case of asystole. Accordingly, in the example illustrated in FIG. 18, it is determined that the waveform of the heart beat signal is not the VF waveform because the pulse signal is effective.

The notification controller may be configured to issue notification related to asystole when it is determined that the pulse signal is ineffective and the waveform of the heart beat signal is likely to be the P wave as the determination result of the determination unit, and issue notification of VF (ventricular fibrillation) when the waveform of the heart beat signal is the VF waveform.

Further, in a case where a QRS shape of the waveform of the heart beat signal is not changed from a QRS shape of a normal wave or the like, there is a high possibility that the SpO2 sensor is detached or the like, the determination unit may be configured to issue notification that the SpO2 sensor is detached instead of issuing the notification related to asystole.

According to the physiological information analysis device 100 of the present embodiment described above, the following effects can be achieved in addition to the effects of the first to third embodiments.

When the heart beat signal is effective and the pulse signal is ineffective, the physiological information analysis device 100 further analyzes the waveform of the heart beat signal, and thus a more appropriate notification can be issued according to the shape (type) of the waveform of the heart beat signal.

### Fifth Embodiment

In the first to fourth embodiments, a case of determining whether the pulse signal is effective has been described. In a fifth embodiment, a case of determining whether a blood pressure signal is effective will be described. In order to avoid redundant description, detailed description of the same configuration as that of the first embodiment will be omitted.

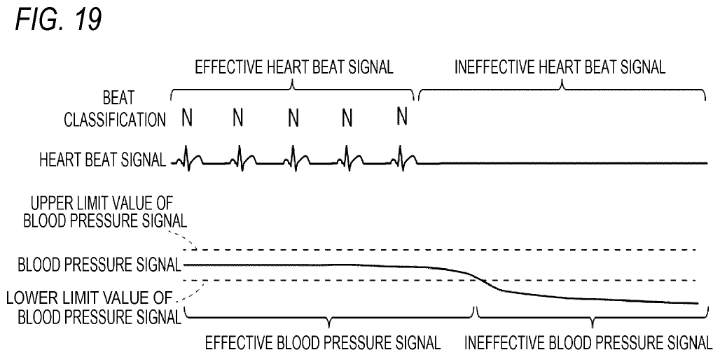

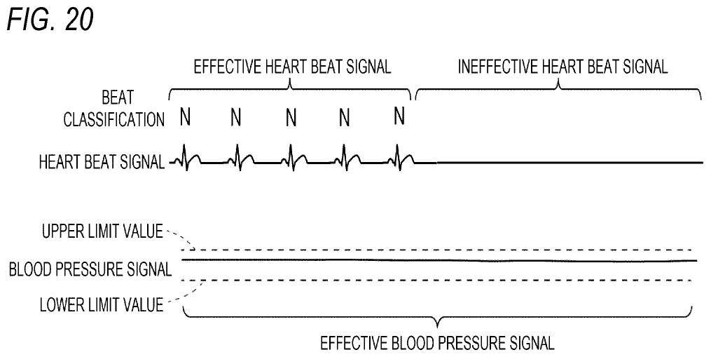

FIG. 19 is a schematic diagram illustrating an example of a correspondence relationship between a heart beat signal and a blood pressure signal in a case where effectiveness of the heart beat signal and effectiveness of the blood pressure signal match in the fifth embodiment, and FIG. 20 is a schematic diagram illustrating an example of the correspondence relationship between the heart beat signal and the blood pressure signal in a case where the effectiveness of the heart beat signal and the effectiveness of the blood pressure signal do not match.

In the present embodiment, the control device 200 obtains a heart beat signal (first physiological information) and a blood pressure signal (second physiological information) of a patient, and determines effectiveness of the heart beat signal and effectiveness of the blood pressure signal. Then, as a result of the effectiveness determination, when the effectiveness of the heart beat signal and the effectiveness of the blood pressure signal match, the control device 200 issues predetermined notification according to the effectiveness, and when the effectiveness of the heart beat signal and the effectiveness of the blood pressure signal do not match, the control device 200 issues the predetermined notification according to the effectiveness of the blood pressure signal. When the obtained blood pressure signal is within a range between an upper limit value and a lower limit value, the blood pressure signal is determined to be effective, and when the obtained blood pressure signal exceeds the upper limit value or falls below the lower limit value, the blood pressure signal is determined to be ineffective.

More specifically, as illustrated in FIG. 19, when both the heart beat signal and the blood pressure signal (mean blood pressure) are effective, the control device 200 does not issue the notification related to asystole, and when both the heart beat signal and the blood pressure signal (mean blood pressure) are ineffective, the control device 200 issues the notification related to asystole.

Further, as illustrated in FIG. 20, when the heart beat signal is ineffective and the blood pressure signal is effective, the control device 200 does not issue the notification related to asystole. As described above, the control device 200 compares the blood pressure signal when the heart beat signal is effective with the blood pressure signal when the heart beat signal is ineffective, and does not issue the notification related to asystole as long as the blood pressure signal is maintained effective and does not change. On the other hand, although not illustrated, when the heart beat signal is effective and the blood pressure signal is ineffective, the control device 200 further analyzes the heart beat signal, and issues notification related to asystole or notification of VF when it is determined that a waveform of the heart beat signal is likely to be a P wave or a VF waveform. On the other hand, when the waveform of the heart beat signal is not the P wave or the VF waveform, sensor abnormality notification is issued instead of issuing the notification related to asystole.

Although the physiological information analysis device, the physiological information analysis method, and the physiological information analysis program according to the embodiments of the presently disclosed subject matter have been described above, the presently disclosed subject matter is not limited to the above-described embodiments.

For example, in the embodiments described above, a case of determining the effectiveness of the pulse signal or the blood pressure signal has been described as an example, but the presently disclosed subject matter is not limited to the case of determining the effectiveness of the pulse signal or the blood pressure signal, and a configuration of determining effectiveness of other physiological information corresponding to the heart beat signal can also be adopted.

In the embodiments described above, a case has been described in which the pulse signal or the blood pressure signal is obtained as the second physiological information, and the effectiveness of the second physiological information is determined, but the presently disclosed subject matter is not limited thereto. For example, the control device 200 may be configured to obtain both the pulse signal and the blood pressure signal as the second physiological information and determine the effectiveness of the second physiological information. Further, the control device 200 may be configured to obtain three or more pieces of physiological information as the second physiological information and determine the effectiveness of the second physiological information.

A part or all of the functions achieved by the physiological information analysis program in the embodiments described above may be achieved by hardware such as an electric circuit.

1. A physiological information analysis device comprising:
an obtaining unit configured to obtain a plurality of pieces of physiological information related to a subject;
a determination unit configured to determine effectiveness of the plurality of pieces of physiological information; and
a notification controller configured to issue, when effectiveness of corresponding first physiological information and second physiological information among the plurality of pieces of physiological information matches, predetermined notification according to the effectiveness, and issue the predetermined notification according to the effectiveness of any physiological information of the first physiological information and the second physiological information when the effectiveness of the first physiological information and the effectiveness of the second physiological information do not match.

2. The physiological information analysis device according to claim 1, wherein
the plurality of pieces of physiological information are selected from a group including information related to a heart beat, information related to a pulse, and information related to a blood pressure.

3. The physiological information analysis device according to claim 1, wherein
the predetermined notification is notification related to asystole or ventricular fibrillation of the subject.

4. The physiological information analysis device according to claim 1, wherein
the first physiological information is a heart beat signal,
the second physiological information is a pulse signal, and
the notification controller issues, when effectiveness of the heart beat signal and effectiveness of the pulse signal match, notification related to asystole of the subject according to the effectiveness, and issues the notification related to asystole of the subject according to the effectiveness of the pulse signal when the effectiveness of the heart beat signal and the effectiveness of the pulse signal do not match.

5. The physiological information analysis device according to claim 4, wherein
as a result of an electrocardiogram analysis on a heart beat of the heart beat signal, when a QRS shape of the heart beat is a predetermined QRS shape, the determination unit determines that the heart beat signal is effective, and determines that the heart beat signal is ineffective when the QRS shape of the heart beat is not the predetermined QRS shape.

6. The physiological information analysis device according to claim 4, further comprising
a mean pulse calculator configured to calculate a mean value of an amplitude of a pulse and a mean value of an interval of the pulse in the pulse signal during a period in which the heart beat signal is effective, wherein
the determination unit determines that the pulse signal is effective when an error of the amplitude of the pulse from the mean value of the amplitude and an error of the interval of the pulse from the mean value of the interval respectively fall within predetermined ranges during a period in which the heart beat signal is ineffective, and determines that the pulse signal is ineffective when any one of the errors falls outside a corresponding one of the predetermined ranges.

7. The physiological information analysis device according to claim 4, further comprising
a reference pulse creation unit configured to create a reference pulse based on a shape of a pulse in the pulse signal during a period in which the heart beat signal is effective, wherein
the determination unit determines that the pulse signal is effective when each of shapes of a predetermined number or more of the pulses matches the shape of the reference pulse during a period in which the heart beat signal is ineffective, the predetermined number being equal to or greater than a first threshold, and determines that the pulse signal is ineffective when each of shapes of a number of the pulses matches the shape of the reference pulse, the number being smaller than the first threshold, or there is no pulse whose shape matches the shape of the reference pulse.

8. The physiological information analysis device according to claim 4, further comprising:
a pulse interval calculator configured to calculate a mean pulse interval of a pulse in a period in which the heart beat signal is effective; and
a pulse interval estimator configured to estimate, based on the mean pulse interval, an appearance timing of the pulse in a period in which the heart beat signal is ineffective, wherein
the determination unit determines that the pulse signal is effective when the pulse appears at the appearance timing during the period in which the heart beat signal is ineffective, and determines that the pulse signal is ineffective when the pulse does not appear at the appearance timing.

9. The physiological information analysis device according to claim 4, further comprising:
a measurement unit configured to measure a delay time from detection of a heart beat to detection of a pulse, wherein
the notification controller determines that the pulse of the pulse signal corresponds to the heart beat of the heart beat signal when the delay time is equal to or less than a predetermined second threshold, and determines that the pulse of the pulse signal does not correspond to the heart beat of the heart beat signal when the delay time exceeds the predetermined second threshold.

10. The physiological information analysis device according to claim 1, further comprising
a log output unit configured to output log information related to the plurality of pieces of physiological information when the effectiveness of the plurality of pieces of physiological information does not match.

11. A physiological information analysis method comprising:
obtaining a plurality of pieces of physiological information related to a subject;
determining effectiveness of the plurality of pieces of physiological information; and
issuing, when effectiveness of corresponding first physiological information and second physiological information among the plurality of pieces of physiological information matches, predetermined notification according to the effectiveness, and issuing the predetermined notification according to the effectiveness of any physiological information of the first physiological information and the second physiological information when the effectiveness of the first physiological information and the effectiveness of the second physiological information do not match.

12. A non-transitory computer readable storage medium storing a physiological information analysis program causing a computer to execute processing comprising:
obtaining a plurality of pieces of physiological information related to a subject;
determining effectiveness of the plurality of pieces of physiological information; and
issuing, when effectiveness of corresponding first physiological information and second physiological information among the plurality of pieces of physiological information matches, predetermined notification according to the effectiveness, and issuing the predetermined notification according to the effectiveness of any physiological information of the first physiological information and the second physiological information when the effectiveness of the first physiological information and the effectiveness of the second physiological information do not match.
